SEQUENCE LISTING (9 sequences)

SEQ ID NO: 1 - protein, 38 aa, Artificial sequence
  Synthetic peptide
HSDGIFTDSYSRYRKQMAVKKYLAAVLGKRYKQRVKNK

SEQ ID NO: 2 - protein, 27 aa, Artificial sequence
  Synthetic peptide
HSDGIFTDSYSRYRKQMAVKKYLAAVL

SEQ ID NO: 3 - protein, 28 aa, Artificial sequence
  Synthetic peptide
HSDAVFTDNYTRLRKQMAVKKYLNSILN

SEQ ID NO: 4 - protein, 28 aa, Artificial sequence
  Synthetic peptide
  MOD_RES (1)..(1): N-acetyl histidine
  MOD_RES (17)..(17): methionine oxide
HSDAVFTDNYTRLRKQMAVKKYLNSILN

SEQ ID NO: 5 - protein, 28 aa, Artificial sequence
  Synthetic peptide
  MOD_RES (1)..(1): N-acetyl histidine
HSDAVFTDNYTRLRKQMAVKKYLNSVLN

SEQ ID NO: 6 - protein, 28 aa, Artificial sequence
  Synthetic peptide
  MOD_RES (1)..(1): N-acetyl histidine
  MOD_RES (17)..(17): methionine oxide
HSDAVFTDNYTRLRKQMAVKKYLNSVLN

SEQ ID NO: 7 - protein, 29 aa, Artificial sequence
  Synthetic peptide
  MOD_RES (28)..(28): 4Abu
HSDGIFTDSYSRYRKQMAVKKYLAAVLEK

SEQ ID NO: 8 - protein, 12 aa, Artificial sequence
  Synthetic peptide
CSKAPKLPAAYC

SEQ ID NO: 9 - protein, 4 aa, Artificial sequence
  Synthetic peptide
CSKC

(19) United States
(12) Patent Application Publication
(10) Pub. No.: US 2021/0055299 A1
(43) Pub. Date: Feb. 25, 2021

(54) METHOD OF USING PACAP AND VIP PEPTIDES FOR DETECTING SHED OR CIRCULATING TUMOR CELLS IN BIOLOGICAL FLUIDS

(71) Applicant: Thomas Jefferson University, Philadelphia, PA (US)

(72) Inventors: Mathew L. THAKUR, Cherry Hill, NJ (US); Leonard G. GOMELLA, Chadds Ford, PA (US)

(21) Appl. No.: 17/089,103

(22) Filed: Nov. 4, 2020

Related U.S. Application Data
(63) Continuation of application No. 14/767,936, filed on Aug. 14, 2015, now abandoned.
(60) Provisional application No. 61/765,329, filed on Feb. 15, 2013.

(51) Int. Cl. G01N 33/574; G01N 33/58

(52) U.S. Cl. CPC G01N 33/574; G01N 33/582

(57) ABSTRACT

This invention relates to a method for detecting shed or circulating tumor cells in a biological fluid using labeled pituitary adenylate cyclase activating peptide or vasoactive intestinal peptide.

### INTRODUCTION

This application is a continuation of U.S. Ser. No. 14/767,936 filed Aug. 14, 2015, which is the National Phase application of PCT/US2014/015758 filed Feb. 11, 2014, which claims the benefit of priority of U.S. Provisional Application No. 61/765,329, filed Feb. 15, 2013, the contents of which are incorporated herein by reference in their entirety.

This invention was made with government support under contract number CA157372-01A1 awarded by the National Institutes of Health. The government has certain rights in the invention.

### BACKGROUND OF THE INVENTION

There is evidence that primary cancers begin shedding neoplastic cells into the circulation at an early disease stage prior to the appearance of clinical manifestations. Malignant tumors are characterized by their ability to invade adjacent tissue. In general, tumors with a diameter of 1 mm are vascularized and animal studies show that as much as 4% of the cells present in the tumor can be shed into the circulation in a 24 hour period (Butler & Gullino (1975) Cancer Res. 35:512-516).

Various methods for isolating and detecting tumor cells in blood have been described. For example, U.S. Pat. No. 6,190,870 teaches immunomagnetic isolation followed by flow cytometric enumeration. However, there is also no visual analysis of the samples. U.S. Pat. No. 6,197,523 describes enumerating cancer cells in 100 μl blood samples. The methods use capillary microscopy to confirm the identity of cells that are found. Further, U.S. Pat. No. 6,365,362 describes methods for immunomagnetically enriching and analyzing samples for tumor cells in blood. In accordance with the methods of this patent, the isolated cells are labeled for the presence of nucleic acid and an additional marker, which allows the exclusion of non-target sample components during analysis. WO 02/20825 describes using an adhesion matrix for enumerating tumor cells. Briefly, the matrix is coated with specific adhesion molecules that will bind to cancer cells with metastatic potential. The matrix can then be analyzed for the presence and type of captured cells.

Recent approaches to drug discovery focus on understanding the genesis of disease and the biomedical pathways controlling disease at a molecular level. Previous studies have demonstrated that VPAC receptors (combined for vasoactive intestinal peptide (VIP) and pituitary adenylate cyclase activating peptide (PACAP)) are overexpressed in high density on BC cells (Reubi, et al. (2000) Cancer Res. 60(11):3105-3112). VPAC receptors are involved in cell proliferation, cell differentiation, as well as in survival of BC cells. On stroma, normal cells, and benign masses only a few VPAC receptors are expressed (Reubi, et al. (2000) supra; Zia, et al. (1996) Cancer Res. 56(15):3486-89; Leyton, et al. (1999) Breast Canc. Res. Treat. 56(2):177-186; Moody & Gozes I (2007) Curr. Pharm. Des. 13(11):1099-1104; Valdehita, et al. (2010) Peptides 31(11):2035-2045; Valdehita, et al. (2012) Mol. Cell Endocrinol. 348(1):241-246).

Radiolabeled biomolecules with high affinities for VPAC receptors have been developed and analyzed in a preclinical setting (Chakder & Rattan (1993) J. Pharm. Expt. Therapeut. 266:392-399; Thakur, et al. (2000) J. Nucl. Med. 41:107-110; Pallela, et al. (1999) J. Nucl. Med. 40(2):352-360; Kolan, et al. (1997) J. Label. Comp. Radiopharmaceut. 40:455-457; Thakur, et al. (2004) J. Nucl. Med. 45:1381-1389; Zhang, et al. (2007) Reg. Peptides 144:91-100; Thakur (2009) Semin. Nucl. Med. 39:236-246; Thakur, et al. (2010) J. Nucl. Med. 51:106-111; Zhang, et al. (2008) J. Nucl. Med. 49:112-121; U.S. Pat. No. 6,855,308). Based on their high affinity for VPAC receptors, peptide constructs, modified by radiolabeling with Tc99m (t½—6 hours, γ—140 keV), have been generated and evaluated for receptor affinity (Kd), receptor specificity, in vivo stability and tissue distribution (Thakur, et al. (2000) supra; Pallela, et al. (1999) supra; Kolan, et al. (1997) supra; Thakur, et al. (2004) supra; Zhang, et al. (2007) supra; Thakur (2009) supra; Thakur, et al. (2010) supra; Zhang, et al. (2008) supra). In addition, peptides have been labeled with β+ (19%, 656 keV) emitting Cu-64 (t½—12.8 hours) for positron emission tomography (PET) with N₂S₂ as a chelating agent. Kd values, tissue distribution studies in athymic nude mice bearing T47D human BC, receptor blocking studies, receptor affinity, and in vivo stability have been examined (Thakur, et al. (2004) supra; Zhang, et al. (2007) supra; Thakur (2009) supra; Thakur, et al. (2010) supra; Zhang, et al. (2008) supra). Of the compounds produced, Cu-64-TP3805 not only imaged all xenografted human BC in athymic nude mice (tumor uptake 6.35±1.28% ID/g at 24 hours post-injection), this compound also localized all (n=8), spontaneously grown BC (visible 5, invisible 1 and metastatic 2) lesions in transgenic MMTVneu mice (n=9) (Thakur, et al. (2010) supra). Furthermore, the Cu-64-TP3805 PET images were normal for two lesions that had negligible expression of VPAC1 receptors. The eight malignant lesions were confirmed by histology and expressed VPAC1 receptors as determined by RT-PCR (Thakur, et al. (2010) supra).

### SUMMARY OF THE INVENTION

The present invention provides methods for detecting shed or circulating tumor cells and diagnosing cancer by contacting a biological fluid with a labeled pituitary adenylate cyclase activating peptide (PACAP) or vasoactive intestinal peptide (VIP), and determining binding of the PACAP or VIP to shed or circulating tumor cells in the biological fluid. In one embodiment, the biological fluid is blood, urine or cerebrospinal fluid.

### DETAILED DESCRIPTION OF THE INVENTION

It has now been found that a labeled pituitary adenylate cyclase activating peptide (PACAP) can bind the VCAP1 receptor and detect tumor cells present in blood or urine. The labeled peptide can detect cells at a concentration of 5 cells/ml sample and correctly identify and distinguish cancer cells from contaminating epithelial cells and white blood cells. The use of this labeled peptide in detecting tumor cells is simple, efficient and does not require expensive equipment or training.

Therefore, the present invention provides methods for detecting tumor cells in a biological fluid and diagnosing cancer using labeled PACAPs or vasoactive intestinal peptides (VIP), both of which bind VPAC1 receptors. In accordance with the present invention, a PACAP is intended to mean a peptide having the sequence of His-Ser-Asp-Gly-Ile-Phe-Thr-Asp-Ser-Tyr-Ser-Arg-Tyr-Arg-Lys-Gln-Met-Ala-Val-Lys-Lys-Tyr-Leu-Ala-Ala-Val-Leu-Gly-Lys-Arg-Tyr-Lys-Gln-Arg-Val-Lys-Asn-Lys (SEQ ID NO:1), or a 29 to 37 amino acid residue fragment thereof. In certain embodiments, the 29 to 37 amino acid residue fragment includes the N-terminal 29 to 37 amino acid residues of the PACAP of SEQ ID NO:1. In particular embodiments, the PACAP has the sequence His-Ser-Asp-Gly-Ile-Phe-Thr-Asp-Ser-Tyr-Ser-Arg-Tyr-Arg-Lys-Gln-Met-Ala-Val-Lys-Lys-Tyr-Leu-Ala-Ala-Val-Leu (SEQ ID NO:2).

A VIP is intended to mean a peptide having the sequence of His-Ser-Asp-Ala-Val-Phe-Thr-Asp-Asn-Tyr-Thr-Arg-Leu-Arg-Lys-Gln-Met-Ala-Val-Lys-Lys-Tyr-Leu-Asn-Ser-Ile-Leu-Asn (SEQ ID NO:3), or derivatives thereof, e.g., His(Ac)-Ser-Asp-Ala-Val-Phe-Thr-Asp-Asn-Tyr-Thr-Arg-Leu-Arg-Lys-Gln-Met(O)-Ala-Val-Lys-Lys-Tyr-Leu-Asn-Ser-Ile-Leu-Asn (SEQ ID NO:4); His(Ac)-Ser-Asp-Ala-Val-Phe-Thr-Asp-Asn-Tyr-Thr-Arg-Leu-Arg-Lys-Gln-Met-Ala-Val-Lys-Lys-Tyr-Leu-Asn-Ser-Val-Leu-Asn (SEQ ID NO:5); His(Ac)-Ser-Asp-Ala-Val-Phe-Thr-Asp-Asn-Tyr-Thr-Arg-Leu-Arg-Lys-Gln-Met(O)-Ala-Val-Lys-Lys-Tyr-Leu-Asn-Ser-Val-Leu-Asn (SEQ ID NO:6), wherein His(Ac) means N-acetyl histidine, and Met(O) means methionine oxide and wherein said peptides have been shown to exhibit the biological activity of VIP (see U.S. Pat. No. 8,329,640).

In some embodiments, one or two of the amino acid residues of the PACAP or VIP have been replaced with a nonstandard amino acid residue. In accordance with this embodiment, a nonstandard amino acid residue includes, but is not limited to, norvaline, norleucine, α-amino-n-butyric acid, α,γ-diaminobutyric acid, γ-aminobutyric acid, ornithine, isovaline, N-ethyl glycine, N-methyl alanine, and isoserine. In certain embodiments, the nonstandard amino acid residue is γ-aminobutyric acid. In particular embodiments, the PACAP has the sequence His-Ser-Asp-Gly-Ile-Phe-Thr-Asp-Ser-Tyr-Ser-Arg-Tyr-Arg-Lys-Gln-Met-Ala-Val-Lys-Lys-Tyr-Leu-Ala-Ala-Val-Leu-γAba-Lys (SEQ ID NO:7).

While peptides binding the VPAC1 receptor are particularly embraced by this invention, it is contemplated that shed or circulating tumor cells in biological fluids can also be detected in accordance with the methods herein using labeled peptides that bind to other markers. For example, binding to carcinoembryonic antigen (CEA) can be used to detect shed prostate or colorectal cancer; CD20 can be used to detect B-cell or non-Hodgkin lymphoma (NHL), chronic lymphocytic leukemia (CLL), and Hodgkin disease (HD); granulocyte-monocyte colony-stimulating factor receptor can be used to detect acute myelogenous leukemia (AML); CD25 can be used in the detection of CD25⁺ T- and B-cell malignancies and cells shed from solid tumors; CD27 can be used in the detection of in indolent NHL; CD40 can be used in multiple myeloma (MM), CLL, mantle cell lymphoma (MCL), and AML; CD44 can be used in NHL and early CLL; CD52 can be used in CLL; CD80 can be used in the detection of CLL and MCL; CD86 in MM and NHL; CD137 in leukemias and lymphomas; CD138 in MM and CLL; CD307 (IRTA2, FcRH5) can be used in the detection of hairy cell leukemia (HCL), MM, CLL, and MCL; beta2-microglobulin in HD, MM, and cells shed from solid tumors; tumor necrosis factor receptor can be used in NHL; mesothelin can be used in the detection of mesotheliomas; CD22 can be used in HCL and insulin-like growth factor 1 receptor (IGFR1) can be used in the detection of prostate cancer. By way of illustration, a labeled Cys-Ser-Lys-Cys (SEQ ID NO:9) or Cys-Ser-Lys-Ala-Pro-Lys-Leu-Pro-Ala-Ala-Tyr-Cys (SEQ ID NO:8) peptide can be used to detect shed or circulating tumor that express IGFR1.

To the PACAP or VIP is coupled a label. A label in accordance with the present invention, is a fluorescent or luminescent molecule that can be detected using conventional microscopic techniques. A label of the invention can be or contain a chromophore, fluorophore or protein. In one embodiment, the fluorescent label may be green fluorescent protein (GFP) or a derivative thereof, e.g., yellow fluorescent protein (YFP), cyan fluorescent protein (CFP), Orange Fluorescent Protein (OFP), enhanced green fluorescent protein (eGFP), modified GFP (emGFP), enhanced yellow fluorescent protein (eYFP); monomeric red fluorescent protein (mRFP); mcherry. In another embodiment, the fluorescent label is fluorescein or rhodamine, as well as fluorescein or rhodamine derivatives, such as fluorescein isothiocyanate (FITC), OREGON (fluorescent dye) Green, Tokyo Green, SNAFL, carboxynaphthofluorescein, ALEXA (fluorescent dye) 488, DYLIGHT (fluorescent dye) 488, tetramethylrhodamine (TAMRA) and its isothiocyanate derivative (TRITC), sulforhodamine 101, Rhodamine Red, ALEXA (fluorescent dye) 546, ALEXA (fluorescent dye) 555, ALEXA (fluorescent dye) 633, DYLIGHT (fluorescent dye) 549, DYLIGHT (fluorescent dye) 633, 4a-diaza-s-indacene (BODIPY), CY-3, CY-5, CY-7, CY-CHROME, R-phycoerythrin (R-PE), etc. Luminescent labels include, but are not limited to, luciferase and aequorin.

In particular embodiments, the label is a near infrared (NIR) fluorophore. For the purposes of this invention, a near-infrared fluorophore includes dyes and other fluorophores with emission wavelengths (e.g., peak emission wavelengths) between about 680 and 1000 nm, e.g., between about 680 and 800 nm, between about 800 and 900 nm, between 700 and 900 nM, between about 900 and 1000 nm, between about 680 and 750 nm, between about 750 and 800 nm, between about 800 and 850 nm, between about 850 and 900 nm, between about 900 and 950 nm, or between about 950 and 1000 nm. Fluorophores with emission wavelengths (e.g., peak emission wavelengths) greater than 1000 nm can also be used in the methods described herein. Exemplary fluorophores include indocyanine green (ICG), IRDYE78, IRDYE80, IRDYE38, IRDYE40, IRDYE41, IRDYE700, IRDYE800, CY5.5, CY7, CY7.5, IR-786, DRAQ5NO (an N-oxide modified anthraquinone), quantum dots, and analogs thereof, e.g., hydrophilic analogs such as sulfonated analogs. Commercially obtainable fluorophores include dyes such as IRDYE78, and IRDYE800CW (LI-COR Biotechnology, Lincoln, Nebr.), ALEXA FLUOR (fluorescent dye) 680, 700, and 750 (Invitrogen, Carlsbad, Calif.), and CYDYE Fluor CY5.5, CY7, AND CY7.5 (GE Healthcare, Chalfont St. Giles, UK). Ideally the fluorochromes are designed to emit at 800±100 nm.

As described herein, the PACAP can be assembled by standard Fmoc chemistry and coupled to the label via conventional chemistries. Likewise, VIP can be synthesized and labeled via conventional chemistries. In one embodiment, the label is coupled to a C-terminal residue of the PACAP or VIP. In another embodiment, the label is coupled to an N-terminal residue of the PACAP or VIP. In a further embodiment, the label is coupled to an amino acid side chain (e.g., lysine). For example, amine-reactive labels (e.g., fluorescent dyes) can be used to modify peptides at the N-terminal or a lysine residue. Three major classes of amine-reactive fluorescent reagents can used to label peptides: succinimidyl esters (SE), isothiocyanates and sulfonyl chlorides.

In addition, amine-containing labels can be used to modify peptides using water-soluble carbodiimides (such as 1-ethyl-3-(3-dimethylaminopropyl)carbodiimide (EDC)) to convert the carboxy groups of the peptides into amide groups. N-hydroxysuccinimide (NHS) may be used to improve the coupling efficiency of EDC-mediated protein-carboxylic acid conjugations. A large excess of the amine-containing labels is usually used for EDC-mediated bioconjugations in concentrated peptide solutions at low pH to reduce intra- and inter-protein coupling to lysine residues.

Because free thiol (SH) groups are not present as abundantly as amino groups in most peptides, thiol-reactive reagents often provide a means of selectively modifying a peptide at a defined site. Therefore thiol-reactive labels can be used to prepare labeled peptides. There are many types of thiol-reactive labels reported in the literature, including iodoacetamides, disulfides, maleimides, vinyl sulfones and various electron-deficient aryl halides and sulfonates. In particular embodiments, the label is functionalized with an iodoacetamide and maleimide moiety. Examples of such labels include, but are not limited to NIR FLUOR780 maleimide, TIDE FLUOR 5 maleimide, and NIR-830 maleimide.

Using the labeled PACAP or VIP described herein, shed or circulating tumor cells can be readily detected in biological fluids. As used herein, shed tumor cells are defined as tumor cells that are released or shed from a primary tumor and transported through the circulatory system to distant sites. The term circulating tumor cells is used herein to mean cells of the blood that are cancerous. For example, blood cancers such as leukemia, lympholma and myeloma are considered circulating tumor cells of this invention.

For the purposes of this invention, a biological fluid is intended to mean a fluid sample obtained from a subject with cancer, a subject suspected of having cancer (e.g., exhibiting one or more symptoms associated with cancer), a subject at risk of having cancer (e.g., because of predisposition or exposure to a carcinogen) or a subject who has been treated for cancer. Desirably, the biological fluid is obtained from a mammal, such as a canine, feline, rodent (e.g., mouse and rat), bovine, ovine, or and primate (e.g., human). In a particular embodiment, the biological fluid is obtained from a human. In a preferred embodiment, the biological fluid is a bodily fluid, including blood, lymph, saliva, mucus, sputum, pus, urine, stool, gastro-intestinal secretions, cochlear fluid, synovial fluid, cerebrospinal fluid, lachrymal fluid, vitreous humor, semen, vaginal secretions, and mammary gland secretions. In a particular embodiment, the biological fluid is blood, urine or cerebrospinal fluid.

Upon contact of a biological fluid with the labeled PACAP or VIP, PACAP binds to VPAC receptors present on the surface of shed or circulating tumor cells, and binding to the shed or circulating tumor cells is detected. In some embodiments, the cells in the biological fluid sample are concentrated (e.g., by centrifugation or filtration) prior to being contacted with the labeled PACAP or VIP. Binding of the labeled PACAP or VIP to shed or circulating tumor cells can be determined by conventional methods including but not limited to fluorescence microscopy, fluorescence microplate reader, flow cytometry, fluorometry, and absorption spectroscopy. Detection may also be by means of an image analysis system utilizing a video camera interfaced to a digitizer or other image acquisition system. Detection may also be by visualization through a filter as under a fluorescence microscope. The microscope may just provide a signal that is visualized by the operator. However, the signal may be recorded on photographic film or using a video analysis system. The signal may also simply be quantified in real-time using either an image analysis system or simply a photometer. In a particular embodiment, binding of the labeled PACAP or VIP to shed or circulating tumor cells is via fluorescent microscopy.

The detection of shed or circulating tumor cells in a biological fluid is of use in disease screening, diagnosing disease, monitoring for recurrence or progression of disease, and/or determining effectiveness of therapeutic intervention (chemical or surgical). Therefore, the present invention also provides a method for diagnosing cancer by contacting a biological fluid obtained from a subject with a labeled PACAP or VIP and determining whether shed or circulating tumor cells are present in the biological fluid. Using this method of the invention, a variety of cancers can be detected and diagnosed including, but not limited to, prostate, bladder, renal, lung, ovarian, breast, pancreatic, throat, uterine, brain, blood or testicular cancer. By way of illustration, shed tumor cells from prostate, bladder and renal cancer can be detected in urine samples, whereas breast, lung and ovarian cancers can be detected in blood samples.

In some embodiments, the diagnostic method of this invention is of use in detecting early stage cancer. As used herein, the phrase “early stage cancer” refers to those cancers, which may have tumors too small to be detected by conventional methods such as mammography for breast cancer patients, or X-rays for lung cancer patients. While mammography can detect tumors having approximately 2×10⁸ cells, the methods of the present invention should enable detection of cancer cells from tumors approximating this size or smaller.

Once the biological fluid from a subject has been contacted with the labeled PACAP or VIP, binding of the labeled PACAP or VIP to cancer cells in the biological fluid is detected, e.g., as described above. The presence or absence of binding of the labeled PACAP or VIP to cancer cells can be assessed by comparing said binding in the test sample to labeled PACAP or VIP binding in a control sample, e.g., a biological fluid known to have shed or circulating tumor cells and/or a biological fluid free of shed or circulating tumor cells. The presence of binding of the labeled PACAP or VIP to shed or circulating tumor cells in the biological fluid is indicative of the subject either having, or being at risk for developing cancer. Thus, a diagnosis of cancer or recurrence of cancer can be made.

The invention will be further described in the following examples, which do not limit the scope of the invention described in the claims.

### Example 1: Confocal Imaging of Prostate Cancer Cells in Urine Samples

The PACAP peptide, His-Ser-Asp-Gly-Ile-Phe-Thr-Asp-Ser-Tyr-Ser-Arg-Tyr-Arg-Lys-Gln-Met-Ala-Val-Lys-Lys-Tyr-Leu-Ala-Ala-Val-Leu-γAba-Lys(R) (SEQ ID NO:7), was synthesized on a Wang resin using an ABI 341A peptide synthesizer (Applied Biosystems, Foster City, Calif.). The 27-amino acid long PACAP sequence was then assembled by standard Fmoc chemistry and coupled with a free sulfhydryl group attached to the C-terminus. This C-terminal sulfhydryl group was then reacted with a maleimide functionalized NIR dye to yield TP4303.

Urine samples from two patients, one with early phase prostate cancer and the other with advanced prostate cancer, were cytospinned, and cells were collected on glass slides. The cells were incubated with TP4303, washed with phosphate-buffered saline (PBS) and the slide was dried. The cells were treated with DAPI (4′, 6-diamidino-2-phenylindole, a fluorescent stain that binds strongly to A-T rich regions in cellular DNA), a cover slip was placed on the sample and confocal microscopic analysis was performed (excitation, 730 μm; emission, 860 μm).

The results of this analysis showed that TP4303 bound to highly dense VPAC1 receptors expressed on the cell membrane of prostate cancer cells from both patients. In addition, while epithelial cells were present, as evidenced by DAPI staining, these cells did not express VPAC1 and did not interact with TP4303. Likewise, while sperm were identified in the sample, as evidenced by DAPI staining of the head, sperm do not express VPAC1 and did not interact with TP4303. Therefore, TP4303 could selectively identify only the cancerous cells and could distinguish the cancerous cells from the normal contaminating cells.

What is claimed is:

1. A method for fluorescently detecting shed prostate tumor cells comprising:
collecting a urine sample from a subject;
concentrating the urine sample;
providing the concentrated urine sample onto a slide;
providing a labeled peptide having a fluorescent label;
incubating the concentrated urine sample with the labeled peptide on the slide;
treating the concentrated urine sample with a DNA stain on the slide;
visualizing the concentrated urine sample on the slide with a microscope;
detecting, with the microscope, binding of the labeled peptide to shed prostate tumor cells in the concentrated urine sample by detecting the label and the DNA stain to both be present on shed prostate tumor cells having VPAC1 receptors that bind pituitary adenylate cyclase activating peptide (PACAP) and vasoactive intestinal peptide (VIP); and
identifying the collected urine sample as having shed prostate tumor cells by the detection of the label and the DNA stain to both be present on cells,
wherein the peptide has the sequence of SEQ ID NO: 2.

2. The method of claim 1, wherein the label has a first fluorescence and the DNA stain has a second fluorescence that is different from the first fluorescence.

3. The method of claim 2, further comprising centrifuging the urine sample to obtain the concentrated urine sample.

4. The method of claim 3, further comprising washing the concentrated urine sample on the slide between the incubation with the labeled peptide and treatment with the DNA stain.

5. The method of claim 4, wherein the collected sample has concentration of cells as low as 5 cells/mL.

6. The method of claim 1, further comprising:
detecting cells that are not shed prostate tumor cells in the concentrated urine sample by detecting the DNA stain to be present on cells without the label being present with the microscope.

7. The method of claim 4, further comprising drying the washed concentrated urine sample prior to treatment with the DNA stain.

8. The method of claim 1, further comprising comparing binding of the labeled peptide to shed prostate tumor cells to binding or lack of binding of labeled peptide in a control sample thereby detecting shed prostate tumor cells in the collected urine sample.

9. The method of claim 1, wherein shed prostate tumor cells are detected with labeled PACAP and said method further comprises:
diagnosing cancer in the subject, wherein the presence of shed prostate tumor cells in the collected urine sample is indicative of the subject having cancer.

10. The method of claim 1, wherein the shed prostate tumor cells having VPAC1 receptors are from a prostate tumor having less than 2×10⁸ cells.
